(19) United States
(12) Patent Application Publication
(10) Pub. No.: US 2025/0237593 A1
(43) Pub. Date: Jul. 24, 2025

(54) Opto-mechanical Analysis Device For Determining Particulate Matter In A Measurement Gas

(71) Applicant: Endress+Hauser SICK GmbH+Co. KG, Ottendorf-Okrilla (DE)

(72) Inventors: Alexander SCHLADITZ, Dresden (DE); Jürgen REGEHR, Dresden (DE); Titus URBAN, Bautzen (DE)

(21) Appl. No.: 19/033,770

(22) Filed: Jan. 22, 2025

(30) Foreign Application Priority Data
Jan. 23, 2024 (EP) 24153448.6

(51) Int. Cl. G01N 15/075; G01N 33/00

(52) U.S. Cl. CPC G01N 15/075; G01N 33/0027

(57) ABSTRACT

An opto-mechanical analysis device for determining particulate matter in a measurement gas includes a first light source, a measurement chamber, a plurality of detectors, a control device, an optical reference measurement element and a displacement unit. A first light of the first light source can be coupled into the measurement chamber. The detectors are configured to receive scattered light that is produced on the incidence of the first light on the particulate matter and to generate scattered light measurement data which is transmitted to the control device. The displacement unit is configured to displace the optical reference measurement element from a parking position into a reference measurement position where the first light can be radiated into the optical reference measurement element. The detectors are configured to receive scattered light from the optical reference measurement element and to generate reference measurement data and to transmit said data to the control device.

The invention relates to an opto-mechanical analysis device for determining particulate matter in a measurement gas, wherein the accuracy of said determination can be continuously checked.

Particulate matter is understood as the smallest particles whose size (aerodynamic diameter) is less than 10 μm.

Particulate matter is characterized as PM10, PM2.5 and PM1. The dust fraction designated as particulate matter PM10 almost exclusively contains particles with a diameter <10 μm. The dust fraction designated as particulate matter PM2.5 almost exclusively contains particles with a diameter <2.5 μm. PM2.5 is thus a subset of PM10. PM1 is to be understood analogously to PM2.5 or PM10.

Particulate matter is particularly critical for public health since in particular the smallest particles (<1 μm) can penetrate deep into the lungs and can even enter the bloodstream. This can lead to a variety of health problems, including respiratory diseases, cardiovascular diseases and lung cancer. The monitoring and reduction of particulate matter emissions is therefore an important part of environmental policy and health protection. The requirements for the determination of particulate matter are constantly increasing since the filter systems of emitting plants, such as coal-fired power stations, are constantly improving so that the concentrations are falling, wherein the permitted emission limits are becoming stricter at the same time.

The particulate matter contained in a measurement gas can be detected using appropriate analysis devices. The problem is that the measurement gas sometimes has high temperatures and the appropriate analysis devices therefore have to be regularly maintained and checked.

It is therefore the object of the present invention here to provide an analysis device for determining particulate matter, which analysis device can be easily checked for its correct function.

The object is satisfied by the opto-mechanical analysis device according to claim 1. Advantageous further developments or embodiments are specified in the dependent claims.

The opto-mechanical analysis device according to the invention serves to determine particulate matter in a measurement gas. The opto-mechanical analysis device comprises at least one first light source, a measurement chamber having an inlet and an outlet, a plurality of detectors, a control device, an optical reference measurement element and a displacement unit. The measurement gas loaded with particulate matter can be supplied to the measurement chamber via the inlet and can be discharged via the outlet. The at least one first light source is configured to generate a first light having a first wavelength. The first light can be coupled into the measurement chamber, where it can interact with the measurement gas. The plurality of detectors are arranged spaced apart from one another at the measurement chamber and are configured to receive scattered light that is produced on the incidence of the first light on the particulate matter. The detectors are further configured to generate scattered light measurement data and to transmit said data to the control device. This transmission can take place by means of an analog signal, such as an analog voltage or an analog current, or by means of a digital signal. Furthermore, the displacement unit is configured to displace (i.e. move) the optical reference measurement element from a parking position into a reference measurement position within the measurement chamber, wherein the first light can be radiated onto the optical reference measurement element in the reference measurement position, and wherein the plurality of detectors are configured to receive the scattered light from the optical reference measurement element in the reference measurement position in order to generate reference measurement data and to transmit said data to the control device.

According to the invention, it is advantageous that an optical reference measurement element is used that can preferably be moved mechanically, in particular from outside the measurement chamber, into the measurement chamber and thus into the light beam of the first light. It can thereby be checked at regular time intervals whether the result of the detectors for the measurement of the reference measurement element changes over time. If the result of the detectors changes, i.e. the reference measurement data change, this is an indication that the opto-mechanical analysis device has a malfunction or that the measurement accuracy has deteriorated significantly. This is because the optical reference measurement element retains its physical properties so that a change in the reference measurement data over time indicates a change in the detectors or the measurement setup.

The opto-mechanical analysis device can generally be operated in a normal operating mode for the actual measurement of particulate matter in the measurement gas. In this normal operating mode, the displacement unit is controlled by the control device such that the optical reference measurement element is in the parking position. In the parking position, the optical reference measurement element is preferably located outside the measurement chamber. In this operating mode, the measurement gas to be analyzed can be introduced into the measurement chamber, wherein the plurality of detectors are configured to generate the corresponding scattered light measurement data and to transmit said data to the control device. On the other hand, the opto-mechanical analysis device can also be operated in a checking mode. In the checking mode, the displacement unit is controlled by the control device such that the optical reference measurement element is in the reference measurement position. In this operating mode, the at least one first light is coupled into the optical reference measurement element so that the plurality of detectors receive scattered light from the optical reference measurement element and generate reference measurement data and transmit said data to the control device.

In an advantageous further embodiment, the opto-mechanical analysis device also comprises a second light source and/or a third light source. The second light source is configured to generate a second light having a second wavelength. The third light source is configured to generate a third light having a third wavelength. The first wavelength, the second wavelength and the third wavelength are selected differently. By means of at least one corresponding mirror, preferably a movable mirror, the light beams of the first, second and third light are brought onto a common light axis so that all the lights can be coupled into the measurement chamber at the same position and are thus incident on the optical reference measurement element.

In an advantageous further embodiment, the wavelength of the first light is 600 to 650 nm. The wavelength of the second light is 900 to 950 nm. The wavelength of the third light is 400 to 450 nm.

In an advantageous further embodiment, a light absorption apparatus is also present that is configured to absorb light coupled into the measurement chamber. A first detector of the detectors is arranged between 0° and 6° directly next to the light absorption apparatus and is in particular configured to detect scattered light in the forward direction. A second detector is arranged at a second scattering angle at 28°. A third detector is arranged at a third scattering angle at 61°. A fourth detector is arranged at a fourth scattering angle at 96°. A fifth detector is arranged at a fifth scattering angle at 128°. A sixth detector is arranged at a sixth scattering angle at 155°. The detectors can thereby detect scattered light at the respective angle.

In an advantageous further embodiment, the displacement unit is configured to displace the optical reference measurement element along a curved movement path from the parking position to the reference measurement position. The optical reference measurement element is preferably not only moved along one axis, but along two axes.

In an advantageous further embodiment, the optical reference measurement element is further away from the inlet of the measurement chamber in the parking position than in the reference measurement position.

In an advantageous further embodiment, the displacement unit is configured to additionally rotate the optical reference measurement element between the parking position and the reference measurement position by approximately or exactly 90°, the rotation preferably taking place about an angle of 60° to 120°. The optical reference measurement element is preferably disposed near a side wall in the parking position and is folded into the measurement chamber to assume the reference measurement position.

In an advantageous further embodiment, the displacement unit comprises a multi-joint mechanism having (preferably at least) one multi-joint connection, a drive motor and a drive shaft, wherein the drive motor is rotationally coupled to the drive shaft. If the drive motor rotates, the drive shaft also rotates. The drive motor is preferably an electric motor, in particular a direct-current motor. A first end of the multi-joint connection is rotationally coupled to the drive shaft. The optical reference measurement element is arranged at a second end of the multi-joint connection, in particular screwed and/or adhesively bonded and/or pressed to this end. A rotary movement of the drive motor in a first direction causes the at least one multi-joint connection to extend and the optical reference measurement element to move from the parking position into the reference measurement position. On the other hand, a rotary movement of the drive motor in a second direction, which is opposite the first direction, causes the at least one multi-joint connection to retract and the optical reference measurement element to move from the reference measurement position into the parking position. It is particularly advantageous that a multi-joint connection is used since the curved movement path for the optical reference measurement element can be realized particularly efficiently via said multi-joint connection.

In an advantageous further embodiment, the displacement unit comprises a detection unit, in particular in the form of a position detection, e.g. a light barrier. The detection unit is configured to detect whether the optical reference measurement element has reached the parking position or the reference measurement position. The detection unit is further configured to output a corresponding detection signal to the control device and/or the drive motor in order to stop the drive motor when the optical reference measurement element has reached the parking position or the reference measurement position.

In an advantageous further embodiment, the detection unit is configured in the form of a position detection, e.g. a light barrier, to recognize various markings that are in particular formed on the drive shaft. A first marking indicates the reaching of the parking position and a second marking indicates the reaching of the reference measurement position. The markings can, for example, be projections that project from the drive shaft in a specific angular position.

In an advantageous further embodiment, a self-locking gear, in particular in the form of a worm gear, is also arranged between the drive motor and the drive shaft. It is thereby ensured that the optical reference measurement element remains in its parking position or its reference measurement position when a drive motor is switched off. The optical reference measurement element is not pushed from its reference measurement position back into its parking position by external influences, for example by an increased pressure of the measurement gas.

In an advantageous further embodiment, the optical reference measurement element comes into contact with the first light only in the reference measurement position, but not in the parking position.

In an advantageous further embodiment, the opto-mechanical analysis device comprises a protective housing that defines a receiving space, wherein the optical reference measurement element is arranged in the receiving space during the parking position. The optical reference measurement element is protected from the measurement gas by such a protective housing.

In an advantageous further embodiment, the protective housing is arranged outside the measurement chamber.

In an advantageous further embodiment, an overpressure unit is provided and is configured to generate an increased air pressure, compared to the environmental pressure, in the receiving space of the protective housing. It is thereby achieved that no measurement gas comprising particulate matter can enter into the protected space and can contaminate the optical reference measurement element.

In an advantageous further embodiment, the opto-mechanical analysis device has a closing cover that is arranged at the optical reference measurement element, wherein the closing cover closes the inlet of the measurement chamber in the reference measurement position. It is thereby ensured that the measurement space is not contaminated by any measurement gas in the checking mode or that the optical reference measurement element is not damaged by hot measurement gas.

In an advantageous further embodiment, the closing cover likewise moves along the curved movement path as soon as the optical reference measurement element moves from the parking position into the reference measurement position and back.

In an advantageous further embodiment, the closing cover moves simultaneously with the optical reference measurement element.

In an advantageous further embodiment, a distance between the closing cover and the optical reference measurement element is always the same and thus constant.

In an advantageous further embodiment, the closing cover is likewise moved by the displacement unit.

In an advantageous further embodiment, the protective housing comprises an opening through which the optical reference measurement element can be moved out of the receiving space and into the receiving space, wherein the closing cover or a further closing cover is provided that closes the opening of the protective housing in the parking position of the optical reference measurement element. It is particularly advantageous that the opening of the protective housing is closed in the parking position of the optical reference measurement element. It is thereby achieved that no measurement gas enters into the protected space and contaminates the optical reference measurement element there. It is furthermore particularly advantageous if it is the same closing cover that also seals the inlet of the measurement chamber in the reference measurement position of the optical reference measurement element since a corresponding sealing of both the protected space and the measurement chamber can be achieved simply by the curved movement path of the optical reference measurement element and the closing cover, and no additional drives are required for this purpose.

In an advantageous embodiment, the closing cover comprises a round cross-section.

In an advantageous embodiment, a cleaning unit is provided and is configured to apply purge air, in particular filtered purge air, to the optical reference measurement element in the reference measurement position. The optical reference measurement element is thereby prevented from being contaminated by the measurement gas or by residues of the measurement gas. The cleaning unit can comprise the overpressure unit or be formed by the overpressure unit.

In an advantageous embodiment, an optical damping element, in particular in the form of a neutral density glass (or also a neutral density filter), is provided and is configured to damp the first light before entering the optical reference measurement element in its reference measurement position. The optical damping element is either attached directly to the optical reference measurement element or in a filter wheel outside the measurement chamber, which filter wheel is irradiated by the at least one first light. The use of such an optical damping element is in particular advantageous since the at least one first light is scattered significantly more strongly at the optical reference measurement element without neutral density glass than at particulate matter that is contained in the measurement gas. Such an optical damping element prevents the detectors from seeing too much scattered light and being damaged or only outputting the maximum value for the brightness (“overload”). The optical damping element is in particular configured to allow only 0.1% of the at least one first light to pass through, i.e. to reduce the light power by a factor of approximately 1000. This damping in particular takes place in the visible spectral range.

In an advantageous embodiment, the damping element is adhesively bonded and/or screwed to the optical reference measurement element.

In an advantageous embodiment, the optical reference measurement element is formed from a glass-ceramic medium or comprises a glass-ceramic medium. The optical reference measurement element is preferably made of Zerodur®. Zerodur® is very homogeneous and has a very low thermal expansion. A reproducible formation of scattered light in the checking mode is thereby possible.

In an advantageous embodiment, the first light has a beam diameter of at least 2 mm before it enters the optical reference measurement element in its reference measurement position. The light beam of the at least one first light can in this respect be expanded via at least one corresponding optics, in particular a lens. Fluctuations in the scattered light at the detector due to possible inhomogeneities in the optical reference measurement element are thereby reduced or excluded.

In an advantageous embodiment, the control device is configured to control the displacement unit such that the latter pivots the optical reference measurement element at regular time intervals from the parking position into the reference measurement position, wherein the control device is further configured to compare reference measurement data that were generated at different points in time with one another and, in the event that a deviation is above a threshold value, to output a signal locally and/or to a higher-ranking control apparatus. The signal can be output locally, for example by an alarm. The alarm can be an optical and/or acoustic alarm. The control device preferably compares reference measurement data that were recorded on different days or months with one another. If there is a deviation above a threshold value, a technician can carry out a check of the opto-mechanical analysis device.

In an advantageous embodiment, a line system that transports the measurement gas loaded with particulate matter in the direction of the inlet of the measurement chamber comprises a tapering in cross-section, in particular in diameter. The tapering only extends over a certain length. Before and after the tapering, the cross-section is again larger than in the region of the tapering.

The invention will be described purely by way of example with reference to the drawings in the following. There are shown:

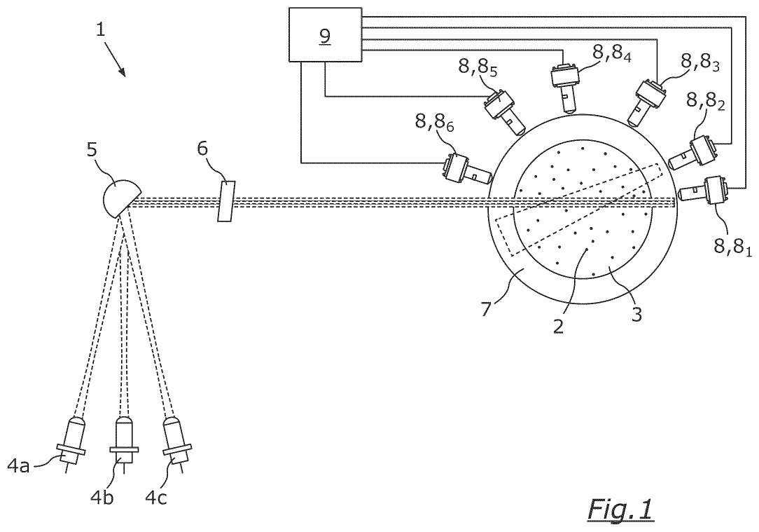

FIG. 1: an embodiment example of an opto-mechanical analysis device according to the invention into whose measurement chamber a measurement gas loaded with particulate matter is introduced;

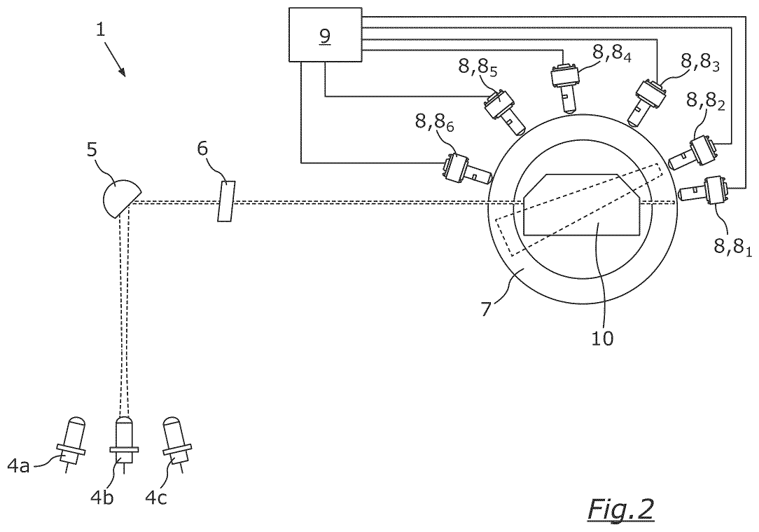

FIG. 2: an embodiment example of the opto-mechanical analysis device according to the invention into whose measurement chamber the optical reference measurement element is pivoted;

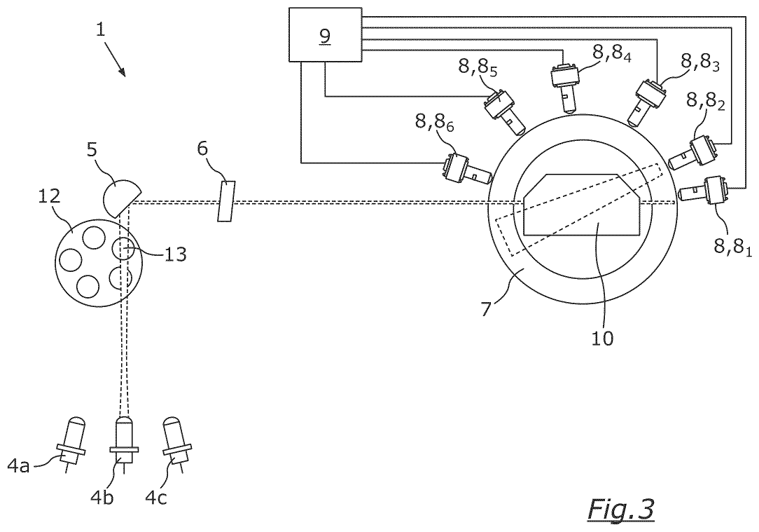

FIG. 3: an embodiment example of the opto-mechanical analysis device according to the invention that describes the damping of a light that is coupled into the optical reference measurement element;

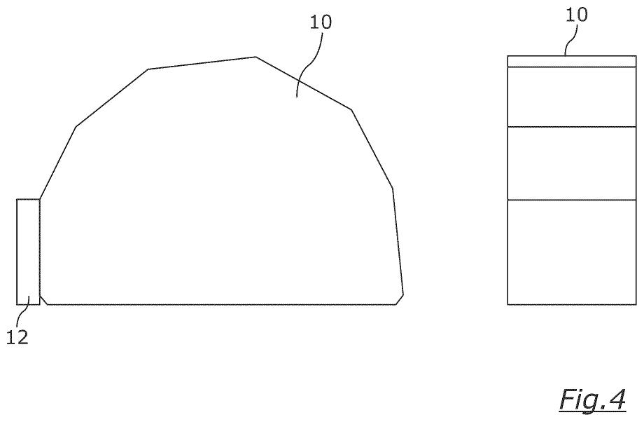

FIG. 4: various representations of the optical reference measurement element;

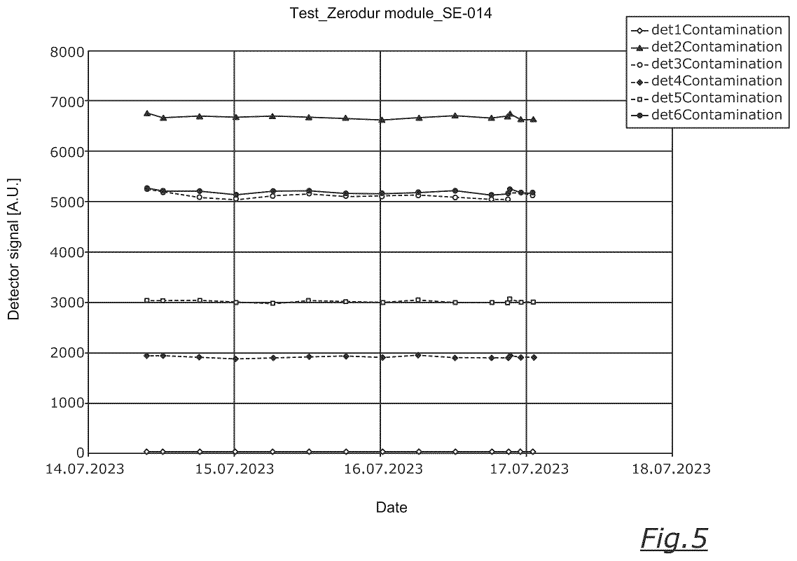

FIG. 5: an exemplary course of reference measurement data that were recorded by the various detectors over a certain time period;

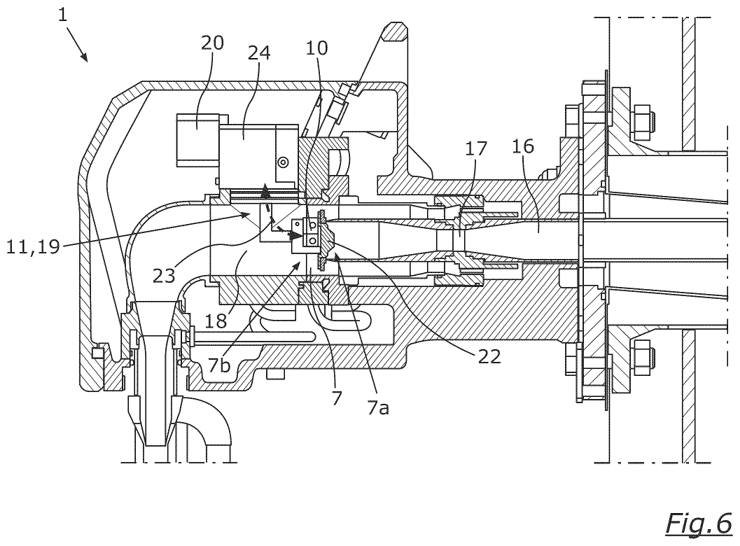

FIG. 6: a section through the opto-mechanical analysis device according to the invention that describes the path of the measurement gas and the position of the optical reference measurement element;

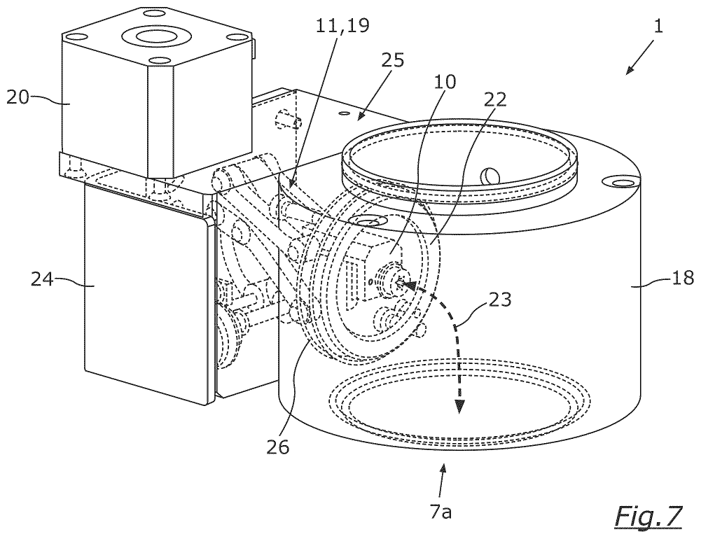

FIG. 7: a spatial representation of a part of the opto-mechanical analysis device;

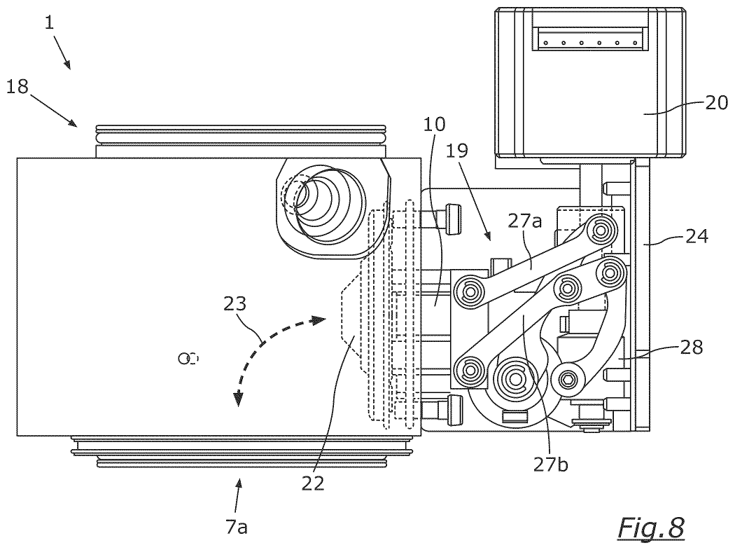

FIG. 8: a spatial representation of a part of the opto-mechanical analysis device; and

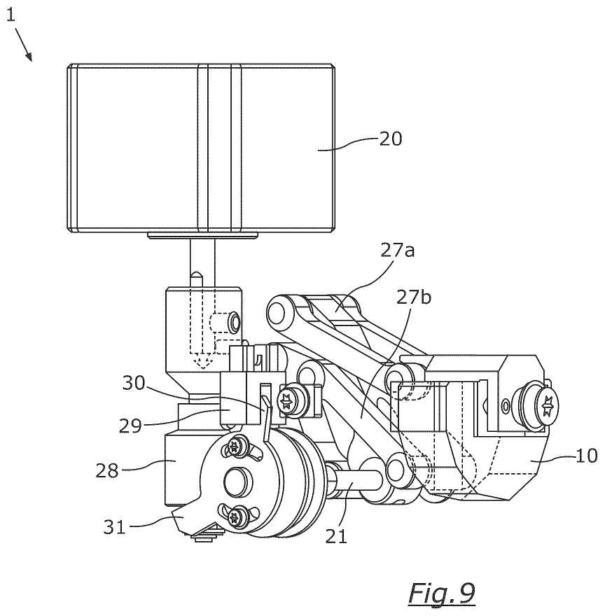

FIG. 9: a spatial representation of a part of the opto-mechanical analysis device to describe the displacement unit.

FIG. 1 shows an embodiment example of an opto-mechanical analysis device 1 that serves to determine particulate matter 2 in a measurement gas 3. In this case, the opto-mechanical analysis device 1 comprises a first light source 4a, a second light source 4b and a third light source 4c. The first light source 4a is configured to generate and transmit light having a first wavelength. The second light source 4b is configured to generate and transmit light having a second wavelength. The third light source 4c is configured to generate and transmit light having a third wavelength. The light beams of the first, second and third light source 4a, 4b, 4c are bundled into a common light beam via a corresponding mirror 5. The at least one light beam or the plurality of light beams can be widened to a certain width via an optics 6. Each light beam in particular has a width of preferably more than 2 mm. The optics 6 can also be directly integrated in the first, second and third light source 4a, 4b, 4c.

The opto-mechanical analysis device 1 furthermore comprises a measurement chamber 7 that has an inlet 7a and an outlet 7b. The measurement gas 3 can be supplied to the measurement chamber 7 via the inlet 7a and the measurement gas 3 can be discharged from the measurement chamber 7 via the outlet 7b.

Furthermore, a plurality of detectors 8 are also provided that are arranged spaced apart from one another at the measurement chamber 7, i.e. at a certain angular distance from one another, and are configured to receive scattered light that is generated on the incidence of the light on the particulate matter 2.

The measurement chamber 7 preferably comprises or consists of metal. The measurement surface 7 has different openings. The detectors 8 are preferably arranged in these openings. Gaps at the openings are preferably sealed in an airtight manner. A further entry opening serves to couple in the light of the at least one first light source 4a. There is preferably also an exit opening via which the coupled-in light is guided out of the measurement chamber 7 again and is in particular led off into an optical sump. Such an optical sump has the property that virtually no light portions are reflected anymore. The optical sump can also be called a light trap.

The optical sump can also be called a light absorption apparatus. The light absorption apparatus is arranged at 0°. A first detector 81 of the detectors 8 is arranged between 0° and 6° directly next to the light absorption apparatus and is in particular configured to detect scattered light in the forward direction. A second detector 82 is arranged at a second scattering angle at 28°. A third detector 83 is arranged at a third scattering angle at 61°. A fourth detector 84 is arranged at a fourth scattering angle at 96°. A fifth detector 85 is arranged at a fifth scattering angle at 128°. A sixth detector 86 is arranged at a sixth scattering angle at 155°. The detectors 8 can thereby detect scattered light at the respective angle.

The measurement chamber 7 preferably has a round cross-section and is further preferably designed as a hollow cylinder. In this respect, the measurement chamber 7 defines a gas receiving space into which the measurement gas 3 is introduced.

The detectors 8 are preferably arranged at the outer wall of the measurement chamber 7. The detectors 18 are preferably arranged in the same plane. The light beam of the at least one first light source 4a preferably also extends in this plane.

Furthermore, the opto-mechanical analysis device 1 also comprises a control device 9. The detectors 8 are configured to receive scattered light and to generate scattered light measurement data in dependence on the brightness and to transmit these scattered light measurement data to the control device 9. This all takes place in a normal operating mode of the opto-mechanical analysis device 1. The control device 9 can also be configured to transmit the scattered light measurement data to a higher-ranking control apparatus that can also be called a guiding device.

FIG. 2 shows a further embodiment example of the opto-mechanical analysis device 1 according to the invention. The opto-mechanical analysis device 1 comprises an optical reference measurement element 10 and a displacement unit 11 (see FIGS. 7, 8, 9). The displacement unit 11 is configured to displace the optical reference measurement element 10 from a parking position into a reference measurement position, wherein the first light can be radiated into the optical reference measurement element 10 in the reference measurement position, and wherein the plurality of detectors 8 are configured to receive scattered light from the optical reference measurement element 10 in the reference measurement position and to generate reference measurement data and to transmit said data to the control device 9. FIG. 2 shows the optical reference measurement element 10 in its reference measurement position, whereas the optical reference measurement element 10 is in its parking position in FIG. 1 and is therefore not shown.

The optical reference measurement element 10 is formed from a glass-ceramic medium or comprises a glass-ceramic medium and is in particular Zerodur®.

FIG. 3 shows a further embodiment example of the opto-mechanical analysis device 1. It is explained that an optical damping element 12 is also provided and is configured to damp the first light before entering the optical reference measurement element 10 in its reference measurement position, wherein the optical damping element 12 is arranged in a filter wheel 13, which is passed through by the first light, outside the measurement chamber 7. This takes account of the circumstance that more light is scattered in the optical reference measurement element 10 in the checking mode than in the normal operating mode in a measurement gas 3 loaded with particulate matter 2. The detectors 8 therefore do not transmit the maximum possible brightness value to the control device 9 or are not damaged.

FIG. 4 shows an embodiment example of the optical reference measurement element 10. The representation at the left-hand side shows a view of the upper side or lower side of the optical reference measurement element 10. The optical reference measurement element 10 is an n-corner, where n≥5. The optical reference measurement element 10 preferably comprises as many corners or angled edges as there are detectors 8. The representation at the right-hand side shows a view of one side of the optical reference measurement element 10. The optical reference measurement element 10 is longer than it is thick. It is furthermore shown that the optical damping element 12 is connected, in particular adhesively bonded, to one side of the optical reference measurement element 10. At least the one first light of the first light source 4a is radiated into the optical reference measurement element 10 via the optical damping element 12.

FIG. 5 shows a variety of reference measurement data that were recorded at different points in time by different detectors 8. The time is plotted on the abscissa and on the coordinate of the detector power. The various measurement value curves with reference measurement data are generated by the different detectors 8. For example, the displacement unit 11 is configured to move the optical reference measurement element 10 from the parking position into the reference measurement position at regular time intervals. In the reference measurement position, the light of the at least one first light source 4a is coupled into the optical reference measurement element 10 so that the plurality of detectors 8 are configured to measure scattered light from the optical reference measurement element 10 and to generate corresponding reference measurement data. It can be seen that there is a jump in the reference measurement data that indicates an anomaly in the opto-mechanical analysis device 1.

FIG. 6 shows a section through the opto-mechanical analysis device 1 according to the invention that describes the path of the measurement gas 3 and the position of the optical reference measurement element 10. The measurement gas 3 loaded with particulate matter 2 is fed to the measurement chamber 7 via a sampling line 16. The sampling line 16 comprises a tapering 17 in cross-section. This tapering 17 only extends over a certain length so that the cross-section is larger before and after this tapering 17 than in the region of the tapering 17. A measurement tube 18 adjoins the outlet 7b of the measurement chamber 7.

In FIG. 6, the optical reference measurement element 10 is located in the measurement chamber 7 and thus in its reference measurement position. The optical reference measurement element 10 is connected to the multi-joint mechanism 19.

The displacement unit 11 comprises a multi-joint mechanism 19, a drive motor 20 and a drive shaft 21 (see FIG. 9). Furthermore, a closing cover 22 is provided that is arranged at the multi-joint mechanism 19 and/or at the optical reference measurement element 10 and, in the reference measurement position shown in FIG. 6, closes the inlet 7a of the measurement chamber 7, in particular in a virtually airtight manner. Measurement gas 3 loaded with particulate matter 2 is thereby prevented from flowing into the measurement chamber 7 in the checking mode and in particular from contaminating the optical reference measurement element 10.

The displacement unit 11 is configured to displace the optical reference measurement element 10 along a curved movement path 23 from the parking position to the reference measurement position and back. The curved movement path 23 is shown by dashed lines in FIG. 6. The displacement unit 11 is configured to rotate the optical reference measurement element 10 by approximately 90° between the parking position and the reference measurement position.

A protective housing 24 that defines a receiving space 25 (see FIG. 7) is furthermore shown in FIG. 6. In the parking position, the optical reference measurement element 10 is located in the receiving space 25 within the protective housing 24. This circumstance is shown in FIG. 7.

FIG. 7 shows an enlarged representation of a part of the opto-mechanical analysis device 1. The measurement chamber 7 is omitted here. The optical reference measurement element 10 is displaced from the receiving space 25 of the protective housing 24 along the movement path 23, in this case through the measurement tube 18 in the direction of the inlet 7a of the measurement chamber 7. At the same time, the closing cover 22 is also displaced in the direction of the inlet 7a of the measurement chamber 7. The closing cover 22 comprises at least one seal, preferably two peripheral seals 26. In the reference measurement position, the closing cover 22 is configured to close the inlet 7a of the measurement chamber 7, preferably in an airtight manner. In the parking position, the same closing cover 22 is configured to close an opening in the protective housing 24, through which opening the optical reference measurement element 10 can be extended from the receiving space 25, in particular into the measurement tube 18. The closing cover 22 in particular likewise closes this opening in a virtually airtight manner.

FIGS. 8 and 9 show a further representation of the opto-mechanical analysis device 1 and in particular explain the mode of operation of the displacement unit 11 and in particular of the multi-joint mechanism 19 in this respect. In this case, the multi-joint mechanism 19 is retracted, which means that the optical reference measurement element 10 is in the parking position.

The drive motor 20 is connected to the drive shaft 21 via a gear 28. The gear 28 is preferably a self-locking gear.

The multi-joint mechanism 19 comprises a first multi-joint connection 27a and a second multi-joint connection 27b. The first multi-joint connection 27a is rotationally coupled to the drive shaft 21 with a first end. If the drive shaft 21 rotates, the first end of the first multi-joint connection 27a also rotates. The second end of the first multi-joint connection 27a is connected to the optical reference measurement element 10. The second multi-joint connection 27b can be rotationally coupled to the drive shaft 21 with its first end or can be rotatably arranged at a stationary part of the multi-joint mechanism 19 or of the protective housing 24. In this case, the force for extending the multi-joint mechanism 19 would be transmitted solely via the first multi-joint connection 27a. A second end of the second multi-joint connection 27b is in turn connected to the optical reference measurement element 10. The optical reference measurement element 10 can be connected to the multi-joint mechanism 19 via a screw connection and/or a clamping connection and/or an adhesive connection.

Both the first multi-joint connection 27a and the second multi-joint connection 27b comprise a plurality of arms that are connected to one another (in series) via a joint.

In particular via the connection of the first multi-joint connection 27a with the drive axle 21 and in interaction with the further pivot points of the entire multi-joint mechanism, it is possible for the optical reference measurement element 10 to follow a curved movement path 23. The second end of the first multi-joint connection 27a and the second end of the second multi-joint connection 27b preferably engage at different points at the optical reference measurement element 10.

The closing cover 22 is not shown in FIG. 9.

The displacement unit 11 furthermore comprises a detection unit 29, in particular in the form of a light barrier, that is configured to detect whether the optical reference measurement element 10 has reached the parking position or the reference measurement position. The detection unit 29 is configured to output a corresponding detection signal to the control device 9 and/or the drive motor 20 in order to stop the drive motor 20.

The detection unit 29 in the form of the light barrier is configured to recognize various markings 30, 31 that are in particular formed on the drive shaft 21. A first marking 30 indicates the reaching of the parking position and a second marking 31 indicates the reaching of the reference measurement position again. The markings 30, 31 can, for example, be projections that project from the drive shaft 21 in a specific angular position.

The invention is not restricted to the embodiment examples described. Within the scope of the invention, all the described and/or drawn features can be combined with one another in any desired manner.

### REFERENCE NUMERAL LIST

opto-mechanical analysis device 1
particulate matter 2
measurement gas 3
first light source 4a
second light source 4b
third light source 4c
mirror 5
optics 6
measurement chamber 7
inlet (measurement chamber) 7a
outlet (measurement chamber) 7b
detectors 8, 81, 82, 83, 84, 85, 86
control device 9
optical reference measurement element 10
displacement unit 11
optical damping element 12
filter wheel 13
sampling line 16
tapering 17
measurement tube 18
multi-joint mechanism 19
drive motor 20
drive shaft 21
closing cover 22
curved movement path 23
protective housing 24
receiving space 25
peripheral seal(s) 26
first multi-joint connection 27a
second multi-joint connection 27b
gear 28
detection unit 29
first marking 30
second marking 31

1. An opto-mechanical analysis device for determining particulate matter in a measurement gas, wherein the opto-mechanical analysis device comprises at least one first light source, a measurement chamber having an inlet and an outlet, a plurality of detectors, a control device, an optical reference measurement element and a displacement unit, wherein
the measurement gas loaded with particulate matter can be supplied to the measurement chamber via the inlet and can be discharged via the outlet, wherein
the at least one first light source is configured to generate a first light having a first wavelength, wherein the first light can be coupled into the measurement chamber, wherein
the plurality of detectors are arranged spaced apart from one another at the measurement chamber and are configured to receive scattered light that is produced on the incidence of the first light on the particulate matter and to generate scattered light measurement data and to transmit said data to the control device, wherein the displacement unit is configured to displace the optical reference measurement element from a parking position into a reference measurement position, wherein the first light can be radiated into the optical reference measurement element in the reference measurement position, and wherein the plurality of detectors are configured to receive scattered light from the optical reference measurement element in the reference measurement position and to generate reference measurement data and to transmit said data to the control device.

2. The opto-mechanical analysis device according to claim 1, wherein the displacement unit is configured to displace the optical reference measurement element along a curved movement path from the parking position to the reference measurement position.

3. The opto-mechanical analysis device according to claim 2, wherein the displacement unit is configured to rotate the optical reference measurement element between the parking position and the reference measurement position by between 60° and 120°.

4. The opto-mechanical analysis device according to claim 1, wherein the displacement unit comprises a multi-joint mechanism having at least one multi-joint connection, a drive motor and a drive shaft, wherein the drive motor is rotationally coupled to the drive shaft, wherein a first end of the at least one multi-joint connection is rotationally coupled to the drive shaft and wherein the optical reference measurement element is arranged at a second end of the at least one multi-joint connection, wherein a rotary movement of the drive motor in a first direction causes the at least one multi-joint connection to extend and the optical reference measurement element to move from the parking position into the reference measurement position, and wherein a rotary movement of the drive motor in a second direction, which is opposite the first direction, causes the at least one multi-joint connection to retract and the optical reference measurement element to move from the reference measurement position into the parking position.

5. The opto-mechanical analysis device according to claim 4, wherein the displacement unit comprises a detection unit that is configured to detect whether the optical reference measurement element has reached the parking position or the reference measurement position, wherein the detection unit is configured to output a corresponding detection signal to the control device and/or the drive motor in order to stop the drive motor.

6. The opto-mechanical analysis device according to claim 5, wherein the detection unit comprises a light barrier.

7. The opto-mechanical analysis device according to claim 4, wherein a self-locking gear is also arranged between the drive motor and the drive shaft, whereby the optical reference measurement element remains in its parking position or its reference measurement position when a drive motor is switched off.

8. The opto-mechanical analysis device according to claim 7, wherein the self-locking gear is a worm gear.

9. The opto-mechanical analysis device according to claim 1, wherein the opto-mechanical analysis device comprises a protective housing that defines a receiving space, wherein the optical reference measurement element is arranged in the receiving space during the parking position.

10. The opto-mechanical analysis device according to claim 5, wherein an overpressure unit is provided and is configured to generate an increased air pressure, compared to the environmental pressure, in the receiving space of the protective housing.

11. The opto-mechanical analysis device according to claim 1, wherein the opto-mechanical analysis device comprises a closing cover that is arranged at the optical reference measurement element, wherein the closing cover closes the inlet of the measurement chamber in the reference measurement position.

12. The opto-mechanical analysis device according to claim 9, wherein the protective housing comprises an opening through which the optical reference measurement element can be moved out of the receiving space and into the receiving space, wherein the closing cover closes the opening of the protective housing in the parking position.

13. The opto-mechanical analysis device according to claim 10, wherein the opto-mechanical analysis device comprises a closing cover that is arranged at the optical reference measurement element, wherein the closing cover closes the inlet of the measurement chamber in the reference measurement position and wherein the protective housing comprises an opening through which the optical reference measurement element can be moved out of the receiving space and into the receiving space, wherein the closing cover closes the opening of the protective housing in the parking position.

14. The opto-mechanical analysis device according to claim 1, wherein a cleaning unit is provided and is configured to blow purge air into the measurement chamber when the optical reference measurement element is in the reference measurement position.

15. The opto-mechanical analysis device according to claim 14, wherein a cleaning unit is provided and is configured to blow filtered purge air into the measurement chamber when the optical reference measurement element is in the reference measurement position.

16. The opto-mechanical analysis device according to claim 1, wherein an optical damping element is provided and is configured to damp the first light before entering the optical reference measurement element in its reference measurement position, wherein the optical damping element is attached directly to the optical reference measurement element or in a filter wheel outside the measurement chamber, which filter wheel is irradiated by the first light.

17. The opto-mechanical analysis device according to claim 16, wherein the optical damping element comprises a neutral density glass.

18. The opto-mechanical analysis device according to claim 1, wherein the optical reference measurement element is formed from a glass-ceramic medium or comprises a glass-ceramic medium.

19. The opto-mechanical analysis device according to claim 18, wherein the optical reference measurement element comprises Zerodur®.

20. The opto-mechanical analysis device according to claim 1, wherein the first light has a beam diameter of at least 2 mm before it enters the optical reference measurement element in its reference measurement position.

21. The opto-mechanical analysis device according to claim 1, wherein the control device is configured to control the displacement unit such that the latter pivots the optical reference measurement element at regular intervals from the parking position into the reference measurement position, wherein the control device is further configured to compare reference measurement data that were generated at different points in time with one another and, in the event that a deviation is above a threshold value, to output a signal locally and/or to a higher-ranking control apparatus.

22. The opto-mechanical analysis device according to claim 3, wherein the displacement unit is configured to rotate the optical reference measurement element between the parking position and the reference measurement position by 90°.
